(19) United States
(12) Patent Application Publication
(10) Pub. No.: US 2023/0082923 A1
(43) Pub. Date: Mar. 16, 2023

(54) METHOD FOR PREPARING EMULSION GEL-BASED FAT SUBSTITUTE WITH ADJUSTABLE PHASE CHANGE AND USE THEREOF

(71) Applicant: Jiangnan University, Wuxi (CN)

(72) Inventors: Zong MENG, Wuxi (CN); Qinbo JIANG, Wuxi (CN)

(21) Appl. No.: 17/989,846

(22) Filed: Nov. 18, 2022

Related U.S. Application Data
(63) Continuation of application No. PCT/CN2022/097373, filed on Jun. 7, 2022.

(30) Foreign Application Priority Data
Jun. 21, 2021 (CN) 2021106873995

(51) Int. Cl. A23D 7/04; A23D 7/01; A23L 29/262; A23L 29/00; A23L 29/275

(52) U.S. Cl. CPC A23D 7/04; A23D 7/01; A23L 29/262; A23L 29/04; A23L 29/275

(57) ABSTRACT

Disclosed is a method for preparing an emulsion gel-based fat substitute with adjustable phase change and use thereof, belonging to the field of oils and fats and food processing. The emulsion gel-based fat substitutes are prepared by using an oil-soluble polysaccharide, oil-soluble small molecule gelling agent, water-soluble large molecule gelling agent and vegetable oil as raw materials, dissolving the oil-soluble polysaccharide, small molecule gelling agent and water-soluble large molecule gelling agent in the heated oil and water phases, mixing and emulsifying the oil solution and aqueous solution to obtain an emulsion, and then gelling the emulsion upon stirring and cooling. An oil-soluble polysaccharide reduces the amount of oil-soluble small molecule gelling agent, an emulsion gel-based fat substitute containing trans-free low-saturated fatty acids is prepared, and the overall oil content of the emulsion gel is reduced by adding the water phase gelled with the water-soluble large molecule gelling agent.

### TECHNICAL FIELD

The present disclosure relates to a method for preparing an emulsion gel-based fat substitute with adjustable phase change and use thereof and belongs to the field of oils and fats and food processing.

### BACKGROUND

The emulsion gels can be subdivided according to the preparation method, raw materials, and composition, such as bicontinuous emulsion gels, oil-in-water emulsion gels, and water-in-oil emulsion gels. The “emulsion gel with adjustable phase change” is an emulsion gel system with adjustable phase change properties, which has the physical properties similar to traditional plastic fats (hydrogenated fats, saturated fats, etc.) that behave as soft solids at room temperature and melt when heated, in which oil-in-water and water-in-oil emulsion gels can be interchanged by changing the ratio of the oil phase to water phase. Emulsion gels are prepared by a combination of emulsification and gelling, such that they contain not only the structural characteristics of emulsions (emulsion droplets and continuous phase) but also the characteristics of gels (gel network structure in the oil and water phases). They combine the excellent properties of emulsions and gels and are expected to be ideal fat substitutes.

Currently, gelation technology is an effective method to reduce trans fatty acids and saturated fatty acids in food products, which, when combined with an emulsification technology to preserve both oil and water phases at the same time, allows the system to be more favorable for use in the substitution of traditional plastic fats. In the gelation of the oil phase, small molecule gelling agents (waxes, glycerides, etc.) are common lipophilic gelation factors that can directly gel the oil phase. However, excessive addition of oil-soluble small molecule gelling agents can cause taste and health problems in the product, thus limiting the application of oleogel. Therefore, oil-soluble large molecule gelation factors can be used to reduce the amount of oil-soluble small molecule gelling agents. Besides, excessive oil intake can aggravate the occurrence of diseases such as obesity. To further reduce oil intake, hydrogel is a good soft solid with plastic fat-like properties that can replace some oil-containing substances to produce low-fat products, and edible protein-based or polysaccharide-based hydrogel agents are beneficial to human health. Emulsion gel provides physical properties similar to solid fat and combines the characteristics of oleogel and hydrogel through the emulsification technology, which reduces the consumption of fats and oils and improves the health and safety of food. In addition, emulsion gels with adjustable phase change can exhibit different physical properties in different states (oil-in-water or water-in-oil), facilitating the adjustment of their physical properties to meet the application requirements.

### SUMMARY

### Technical Problem

Excessive addition of oil-soluble small molecule gelling agents in gelation technology can cause taste and health problems in products; and the emulsion gel-based fat substitutes obtained by other technologies have high fluidities and weak solid-state characteristics.

### Technical Solution

In order to solve at least one of the above problems, thee mulsion gel-based fat substitutes according to the present disclosure are prepared by using an oil-soluble polysaccharide, an oil-soluble small molecule gelling agent, a water-soluble large molecule gelling agent, and a vegetable oil as raw materials, dissolving the oil-soluble polysaccharide, small molecule gelling agent and water-soluble large molecule gelling agent in the heated oil phase and water phase, respectively, mixing and emulsifying the oil solution and the aqueous solution to obtain an emulsion, and then gelling the emulsion upon stirring and cooling. According to the present disclosure, an oil-soluble polysaccharide is introduced into the system to reduce the amount of oil-soluble small molecule gelling agent, an emulsion gel-based fat substitute containing trans-freelow-saturated fatty acids is prepared for replacing the conventional plastic fat, and the overall oil content of the emulsion gel is reduced by adding the water phase gelled with the water-soluble large molecule gelling agent. In addition, the prepared emulsiongel-based fat substitute as adjustable phase change characteristics for different application scenarios, allowing the emulsion gel to be more widely used in food products and providing a reference for the substitution of hydrogenated fats and saturated fats in food products.

A first object of the present disclosure is to provide a method for preparing an emulsion gel-based fat substitute with adjustable phase change, including the steps of:

(1) dissolving an oil-soluble polysaccharide and an oil-soluble small molecule gelling agent in a vegetable oil to obtain an oil solution;
(2) dissolving a water-soluble large molecular gelling agent in water to obtain an aqueous solution; and
(3) evenly mixing the oil solution in step (1) and the aqueous solution in step (2) for emulsification to obtain an emulsion; and gelling the emulsion to obtain an emulsion gel-based fat substitute,
wherein when the mass percentage of the oil solution in the emulsion in step (3) is less than 44% and more than 0, an oil-in-water emulsion gel-based fat substitute is obtained; when the mass percentage of the oil solution in the emulsion in step (3) is more than 44% and less than 100%, a water-in-oil emulsion gel-based fat substitute is obtained; and when the mass percentage of the oil solution in the emulsion in step (3) is equal to 44%, a semi-bicontinuous emulsion gel-based fat substitute (containing both oil-in-water emulsion and water-in-oil emulsion) is obtained.

In one embodiment of the present disclosure, when the mass ratio of the oil solution to the aqueous solution in step (3) is 2:8 to 4:6, the oil-in-water emulsion gel-based fat substitute is obtained; and when the mass ratio of the oil solution to the aqueous solution in step (3) is 5:5 to 8:2, the water-in-oil emulsion gel-based fat substitute is obtained.

In one embodiment of the present disclosure, the oil-soluble polysaccharide in step (1) is one or both of ethyl cellulose and chitin.

In one embodiment of the present disclosure, the oil-soluble small molecule gelling agent in step (1) is one or more of mono- and diglycerides of fatty acid, polyglycerol fatty acid ester, sodium stearoyllactate, and sucrose fatty acid esters.

In one embodiment of the present disclosure, the vegetable oil in step (1) is one or more of peanut oil, soybean oil, sunflower oil, rapeseed oil, corn oil, tea seed oil, sesame oil, olive oil, wheat germ oil, palm oil, hemp seed oil, coconut oil, palm kernel oil, and coconut kernel oil.

In one embodiment of the present disclosure, the water-soluble large molecular gelling agent in step (2) is one or more of gelatin, hydroxypropylmethylcellulose, methylcellulose, and konjacgum.

In one embodiment of the present disclosure, the mass concentration of the water-soluble large molecular gelling agent in the water in step (2) is 4% to 10%.

In one embodiment of the present disclosure, the mass concentration of the oil-soluble polysaccharide in the vegetable oil in step (1) is 5% to 10%.

In one embodiment of the present disclosure, the mass concentration of the oil-soluble small molecule gelling agent in the vegetable oil in step (1) is 1% to 5%.

In one embodiment of the present disclosure, the emulsification in step (3) is a high-speed shearing at 65-80° C. and 5000-15000 rpm for 1-5 min.

In one embodiment of the present disclosure, the gel in step (3) is obtained by stirring the obtained emulsion at 15-30° C. (room temperature) and 100-1000 rpm.

In one embodiment of the present disclosure, the method for preparing an emulsion gel-based fat substitute with adjustable phase change includes the steps of:

(1) dissolving the oil-soluble polysaccharide and the oil-soluble small molecule gelling agent in the vegetable oil at 130-180° C. to obtain the oil solution;
(2) dissolving the water-soluble large molecular gelling agent in the water at 65-80° C. to obtain the aqueous solution; and
(3) evenly mixing the oil solution in step (1) and the aqueous solution in step (2), and emulsifying via a high-speed shearing at 65-80° C. and 5000-15000 rpm for 1-5 min to obtain the emulsion; and then stirring and gelling at 15-30° C. and 100-1000 rpm to obtain the emulsion gel-based fat substitute.

A second object of the present disclosure is an oil-in-water emulsion gel-based fat substitute and a water-in-oil emulsion gel-based fat substitute prepared by the method according to the present disclosure.

A third object of the present disclosure is use of the oil-in-water emulsion gel-based fat substitute and the water-in-oil emulsion gel-based fat substitute according to the present disclosure in the food industry.

In one embodiment of the present disclosure, the use includes the use in the flower shaped cream, 3D printing, embedding of active substances, and customization of food products.

In one embodiment of the present disclosure, the use includes the use of the oil-in-water emulsion gel-based fat substitute in the flower shaped cream, and the mass ratio of the oil solution to the aqueous solution is 4:6 in the method for preparing an oil-in-water emulsion gel-based fat substitute.

In one embodiment of the present disclosure, the use includes the use of the water-in-oil emulsion gel-based fat substitute in the 3D printing, specifically the use of the water-in-oil emulsion gel-based fat substitute as an edible solid material in 3D printing; and the mass ratio of the oil solution to the aqueous solution is 6:4 to 8:2 in the method for preparing water-in-oil emulsion gel-based fat substitute.

In one embodiment of the present disclosure, the use in the embedding of active substances specifically includes the use of the oil-in-water emulsion gel-based fat substitute or the water-in-oil emulsion gel-based fat substitute as a carrier in the embedding and controlled-release of a water-soluble active substance and a fat-soluble active substance.

### Beneficial Effect

(1) The emulsion gel-based fat substitute of the present disclosure is a soft colloidal substance having gel characteristics, exhibiting a semi-solid nature, and possessing emulsion properties, which can be used in food production for the replacement of conventional plastic fats consisting of trans fats and highly saturated fats, and in the pharmaceutical, cosmetic and other industries as a carrier for the embedding and controlled release of functional active substances, etc.

(2) The emulsion gel-based fat substitute of the present disclosure is obtained by using the oil-soluble polysaccharide and oil-soluble small molecule gelling agent as the gelation factor of the oil phase and using water-soluble large molecule gelling agent as the gelation factor of the water phase to prepare an emulsion gel with adjustable phase change, in which the ratio of oil to water phases of the emulsion gel can be changed to adjust the type of emulsion gel according to the application scenario so as to meet specific application requirements.

(3) The method for preparing an emulsion gel-based fat substitute of the present disclosure as a wider range of applications and more flexible application methods than ordinary oleogel, and is also faster and more convenient than the production of conventional plastic fats.

(4) Compared with the oleogel obtained by using oil-soluble small molecule gelling agent alone, the emulsion gel-based fat substitute of the present disclosure is obtained by using the oil-soluble polysaccharide, oil-soluble small molecule gelling agent, and water-soluble large molecule gelling agent significantly reduces the amount of oil-soluble small molecule gelling agent and the amount of vegetable oil (the amount of vegetable oil can be reduced to 10% of the system, i.e. 90% of the oil phase is replaced by the water phase) under the action of oil-soluble polysaccharide and water-soluble large molecule gelling agent, and the phase change of the emulsion gel-based fat substitute can be controlled by changing the ratio of oil to water phases to adjust the physical properties of the emulsion gel-based fat substitute, in which the emulsion gel-based fat substitute is in the form of oil-in-water when the mass percentage of oil solution is lower than 44%, and the emulsion gel-based fat substitute is in the form of water-in-oil when the mass percentage of oil solution is higher than 44%. These advantages facilitate the customization of the emulsion gel-based fat substitute to meet the performance requirements of different application scenarios.

(5) The emulsion gel-based fat substitute of the present disclosure is fast to produce, simple to process, and exhibits physical properties under different types in line with a variety of application scenarios; and the oil-in-water emulsion gel-based fat substitute can be used for the flower shaped cream, and the water-in-oil emulsion gel-based fat substitute can be used for the 3D printing.

(6) The emulsion gel-based fat substitute of the present disclosure is obtained using polysaccharides of plant and animal origin as raw materials, which greatly reduces the amount of oil-soluble small molecule gelling agent and has the environmental protection concept of green and sustainable food development.

(7) The emulsion gel-based fat substitute of the present disclosure exhibits good viscoelasticity, has obvious thixotropy, and can quickly recover not less than 70% of modulus after being subjected to deformation, and has good plasticity.

(8) The emulsion gel-based fat substitute of the present disclosure has a better embedding and slow release effect on both water-soluble and fat-soluble functional active components, which greatly delays the release rate of the functional components.

(9) The emulsion gel-based fat substitute of the present disclosure contains low content of saturated fatty acids and no trans fatty acids, and can be used for replacing traditional plastic fats in meat products, as well as for preparing customized food products, which are green, healthy, customized, and rich in functionality.

(10) In the present disclosure, oil-soluble polysaccharides construct a network structure in oil solution, and water-soluble large molecules construct a network structure in aqueous solution; and oil-soluble small molecules act as interfacial stabilizers at the oil-water interface, so that the oil and water phases are mixed evenly to further form a double network structure, which is completely different from the network structure constructed by small molecules and lecithin at the oil-water interface, which is currently disclosed in some literature.

### BRIEF DESCRIPTION OF FIGURES

FIG. 1A shows the elastic modulus (G′) and viscous modulus (G″) under strain scanning for the two emulsion gel-based fat substitutes in Examples 1 and 2, which are prepared at a mass ratio of the oil solution to the aqueous solution of 4:6 and 6:4, respectively.

FIG. 1B shows the elastic modulus (G′) and viscous modulus (G″) under frequency scanning for the two emulsion gel-based fat substitutes in Examples 1 and 2, which are prepared at a mass ratio of the oil solution to the aqueous solution of 4:6 and 6:4, respectively.

FIG. 1C shows the elastic modulus (G′) and viscous modulus (G″) under temperature scanning for the two emulsion gel-based fat substitutes in Examples 1 and 2, which are prepared at a mass ratio of the oil solution to the aqueous solution of 4:6 and 6:4, respectively.

FIG. 1D shows the elastic modulus (G′) and viscous modulus (G″) under time scanning for the two emulsion gel-based fat substitutes in Examples 1 and 2, which are prepared at a mass ratio of the oil solution to the aqueous solution of 4:6 and 6:4, respectively.

FIG. 2A shows the schematic diagram of oil-in-water emulsion gel-based fat substitute prepared in Example 1.

FIG. 2B shows the schematic diagram of a semi-bicontinuous emulsion gel-based fat substitute prepared in Example 3.

FIG. 2C shows the schematic diagram of water-in-oil emulsion gel-based fat substitute prepared in Example 2.

FIG. 3 shows laser confocal micrographs for the two emulsion gel-based fat substitutes in Examples 1 and 2, in which oil and glycerol monostearate are stained and overlaid, and the emulsion gel-based fat substitutes are in the forms of oil-in-water (oil solution:aqueous solution=4:6) and water-in-oil (oil solution:aqueous solution=6:4), respectively.

FIG. 4A shows the infrared spectra of ethyl cellulose powder, gelatin powder, and glycerol monostearate powder.

FIG. 4B shows the infrared spectra of the emulsion gel-based fat substitutes in Examples 1 and 2.

FIG. 5 shows the hardness for the emulsion gel-based fat substitutes obtained at different ratios of the oil solution to the aqueous solution in Example 4, the oleogel made of glycerol monostearate and ethylcellulose, and the hydrogel made of gelatin.

FIG. 6A shows a confocal micrograph of Comparative Example 1.

FIG. 6B shows a confocal micrograph of Comparative Example 2.

FIG. 6C shows a physical view of Comparative Example 3.

FIG. 6D shows the oil leakage rate of Comparative Example 4.

FIG. 7 shows the appearance of the flower shaped cream prepared from the emulsion gel-based fat substitute (oil-in-water type) in Example 5.

FIG. 8 shows the 3D printing effect of the emulsion gel-based fat substitute (water-in-oil type) in Example 6.

FIG. 9A shows the release rate of vitamin C in hydrogel made of gelatin (10%).

FIG. 9B shows the release rate of vitamin C in the emulsion gel-based fat substitute.

FIG. 9C shows the release rate of vitamin E in the oleogel made of glycerol monostearate and ethyl cellulose (2% glycerol monostearate and 10% ethyl cellulose).

FIG. 9D shows the release rate of vitamin E in the emulsion gel-based fat substitute.

### DETAILED DESCRIPTION

Preferred examples of the present disclosure are described below, it is understood that the examples are intended to better explain the present disclosure and are not intended to limit the present disclosure. The parts in the examples are all parts by mass.

Test Method:

1. Rheological properties of the emulsion gel-based fat substitute: The test is performed by a DHR-3 rotational rheometer, and the elastic modulus G′ and viscous modulus G″ are measured using a 40 mm diameter steel plate. The amplitude scanning, frequency scanning, temperature scanning and time scanning are performed at 25° C., with the strain ranging from 0.01% to 100% in the amplitude scanning, the frequency ranging from 0.1 Hz to 10 Hz in the frequency scanning, the temperature ranging from 25° C. to 70° C. in the temperature scanning, and the strain varying repeatedly between 100% and 0.01% for 30 s at each stage in the time scanning.

2. Texture of the emulsion gel-based fat substitute, oleogel made of glycerol monostearate and ethyl cellulose, and hydrogel made of gelatin: The test is performed by a TAXT texturizer, and the hardness of the samples is measured by a single compression test using P/25 probe with the pre-test, test, and post-test speeds of 5 mm/s, 1 mm/s, and 5 mm/s and the strain degree of 30%.

3. Laser confocal microscope (LSM-880) is used to observe the microstructure of the emulsion gel-based fat substitute.

4. The infrared spectral data for raw material powder and lyophilizede mulsion gel-based fat substitute are measured by Fourier transform infrared spectrometer (Nicolet iS-10), and the spectral data within the wave number range of 4000-600 cm⁻¹ are collected by iTR attachment.

### Example 1

A method for preparing an emulsion gel-based fat substitute with adjustable phase change includes the steps of:

(1) 10 parts of ethyl cellulose and 2 parts of glycerol monostearate were weighed and dissolved in 88 parts of soybean oil at 150° C., stirred for 10 min and then placed in a water bath at 70° C. to obtain an oil solution;

(2) 10 parts of gelatin were dissolved in 90 parts of hot water at 70° C. and stirred for 10 min to obtain an aqueous solution; and

(3) the oil solution in step (1) and the aqueous solution in step (2) were mixed evenly according to the mass ratio of 4:6, and the mixed solution was emulsified for 2 min at a rate of 10,000 rpm using a high-speed homogenizer to obtain an emulsion; and then the obtained emulsion was stirred at room temperature at a low speed of 400 rpm until gelling the system to obtain an oil-in-water emulsion gel-based fat substitute.

### Example 2

A water-in-oil emulsion gel-based fat substitute was obtained according to the method in Example 1, except for adjusting the mass ratio of oil solution to aqueous solution in step (3) of Example 1 as 6:4.

### Example 3

A semi-bicontinuous emulsion gel-based fat substitute (containing both oil-in-water emulsion and water-in-oil emulsion) was obtained according to the method in Example 1, except for adjusting the mass ratio of oil solution to aqueous solution in step (3) of Example 1 as 44:56.

The emulsion gel-based fat substitutes obtained in Examples 1-3 were tested for performance and the test results were as follows:

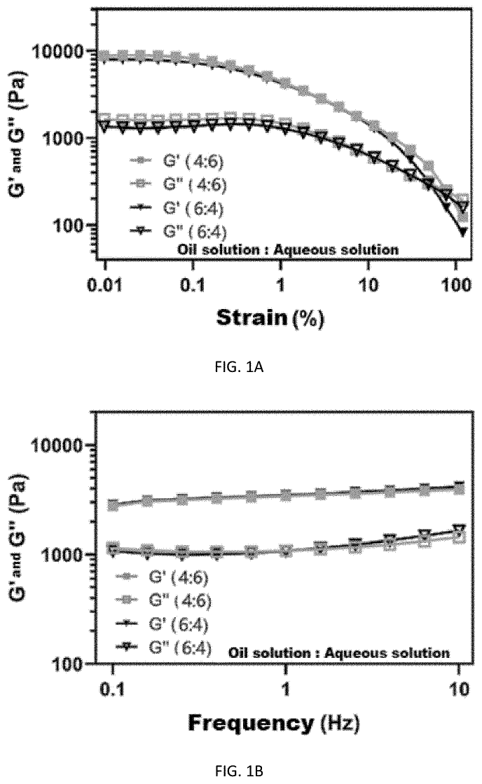

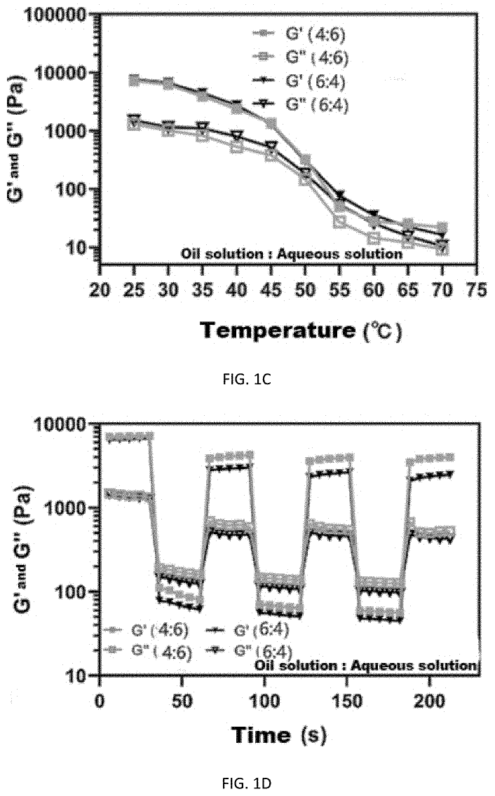

FIGS. 1A, 1B, 1C, and 1D show the rheological data of the two emulsion gel-based fat substitutes in Examples 1 and 2. It can be seen from FIGS. 1A, 1B, 1C, and 1D that: in the strain scanning, the two emulsion gel-based fat substitutes had basically the same elastic modulus, and the emulsion gel-based fat substitute (water-in-oil type) with the oil solution/aqueous solution of 6:4 had a slightly lower viscous modulus, indicating that the water-in-oil emulsion gel-based fat substitute had a higher fluidity; in the frequency scanning, the two emulsion gel-based fat substitutes had basically the same elastic modulus and viscous modulus, the elastic modulus was much larger than the viscous modulus, and the two emulsion gel-based fat substitutes both behaved as viscoelastic semisolids; in the temperature scanning, the two emulsion gel-based fat substitutes had reduced elastic modulus and viscous modulus during the temperature increase, the modulus decreased rapidly at 45° C., and the elastic modulus decreased to the same as the viscous modulus at 70° C., indicating that the two emulsion gel-based fat substitutes had disappeared viscoelasticity and higher fluidities, and became liquid mixtures; in the time scanning, the two emulsion gel-based fat substitutes had rapidly decreased modulus when subjected to high strain shear, indicating that the two emulsion gel-based fat substitutes had significant thixotropy and a greater degree of modulus recovery (not less than 70%) after removing the high strain, and the emulsion gel-based fat substitute (oil-in-water type) with oil solution:aqueous solution=4:6 had a greater degree of modulus recovery and better structural recovery; and the two emulsion gel-based fat substitutes had a less rheological property difference and mainly behaved as semi-solids with viscoelasticity, which facilitates their use as substitutes for plastic fats in food products.

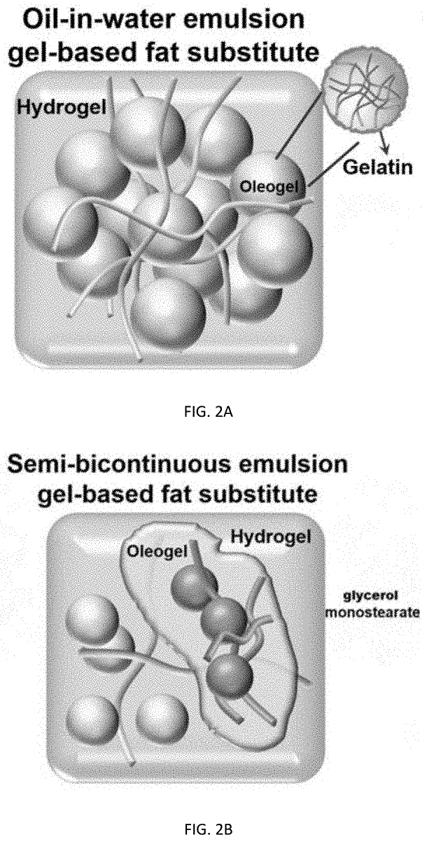

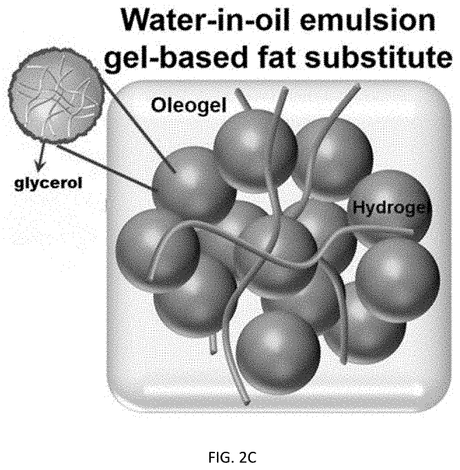

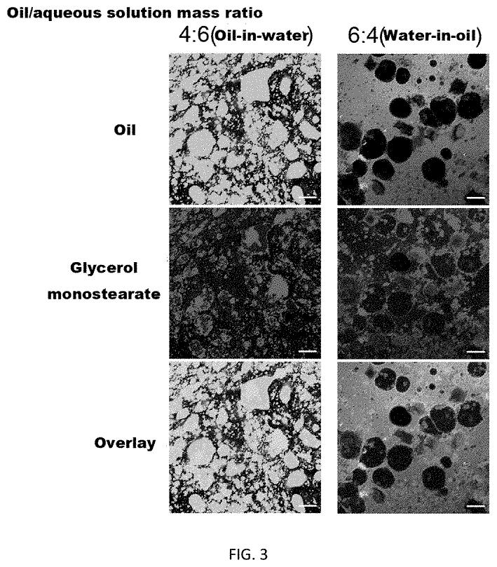

FIGS. 2A, 2B and 2C are schematic diagrams showing the phase change phenomenon of the emulsion gel-based fat substitutes in Examples 1, 2 and 3 at different ratios of the oil solution to the aqueous solution. FIG. 3 shows the laser confocal micrographs of the two emulsion gel-based fat substitutes in Examples 1 and 2. When the mass ratio of oil solution to aqueous solution was 4:6, the emulsion gel-based fat substitute was in the form of oil-in-water, and thus the oil was the dispersed phase dispersed in the system; when the mass ratio of oil solution to aqueous solution was 6:4, the emulsion gel-based fat substitute was in the form of water-in-oil, and thus the oil was the continuous phase surrounding the dispersed system, since glycerol monostearate was fat-soluble, its distribution was basically the same as that of oil; and when the mass ratio of oil solution to aqueous solution is 44:56, the oil-in-water emulsion and water-in-oil emulsion were contained at the same time.

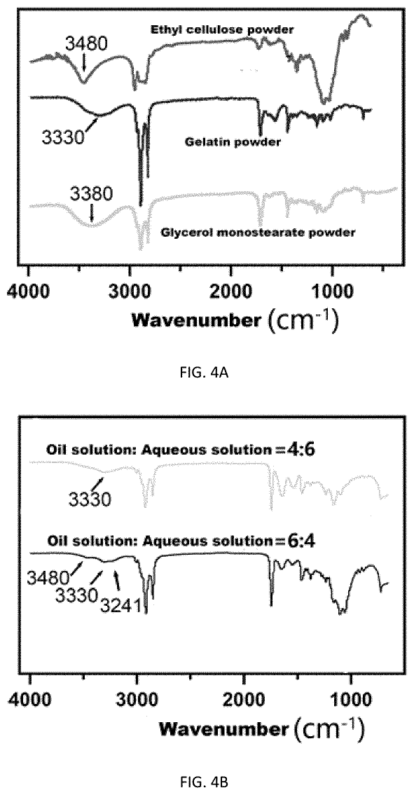

FIGS. 4A and 4B show the infrared spectra of ethyl cellulose powder, gelatin powder, and glycerol monostearate powder, as well as the infrared spectra of the two emulsion gel-based fat substitutes in Examples 1 and 2. When the mass ratio of oil solution to aqueous solution was 4:6, the emulsion gel-based fat substitute was in the form of an oil-in-water, and the outer phase was hydrogel made of gelatin at this time. It can be seen from the infrared spectra of this emulsion gel-based fat substitute that the presence of a large amount of gelatin led to only one characteristic peak within the wave number range of 3200-3500 cm⁻¹, corresponding to the N—H stretching vibration absorption peak of gelatin powder at 3330 cm⁻¹. Apparently, the N—H stretching vibration absorption peak of gelatin masked the hydroxyl stretching vibration absorption peaks of ethyl cellulose and glycerol monostearate. When the mass ratio of oil solution to aqueous solution was 6:4, the emulsion gel-based fat substitute was in the form of water-in-oil, and the outer phase was oleogel at this time. It can be seen from the infrared spectra of this emulsion gel-based fat substitute that three peaks appeared in the wave number range of 3200-3500 cm⁻¹, namely the hydroxyl stretching vibration absorption peak of ethyl cellulose at 3480 cm⁻¹, the N—H stretching vibration absorption peak of gelatin at 3330 cm⁻¹, and the hydroxyl stretching vibration absorption peak of glycerol monostearate at 3241 cm⁻¹. Since hydrogen bonds were formed by glycerol monostearate in oil, the corresponding hydroxyl stretching vibration absorption peaks were red-shifted in the emulsion gel-based fat substitute.

### Example 4

An emulsion gel-based fat substitute was obtained according to the method in Example 1, except for adjusting the mass ratio of oil solution to aqueous solution in step (3) of Example 1 as 2:8, 4:6 (Example 1), 5:5, 6:4 (Example 2), and 8:2.

The emulsion gel-based fat substitutes as obtained were tested for performance and the test results were as follows:

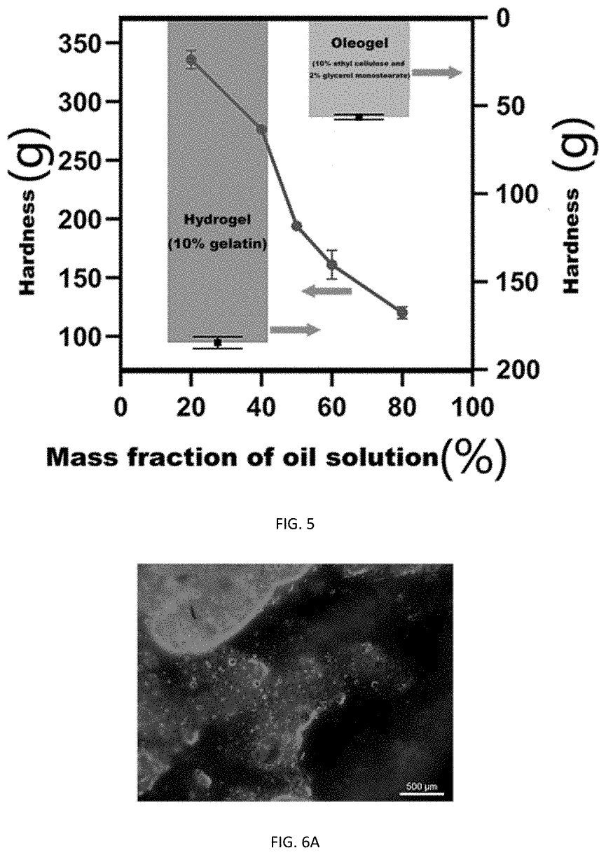

FIG. 5 shows the hardness of the emulsion gel-based fat substitute obtained at different ratios of the oil solution to the aqueous solution, as well as the hardness of the oleogel containing 10% ethyl cellulose and 2% glycerol monostearate, and the hardness of the hydrogel containing 10% gelatin. It can be seen from FIG. 5 that the hardness of the emulsion gel-based fat substitute shada gradual decrease with increasing mass fraction of oil solution. Although the rheological properties of the emulsion gel-based fat substitutes with oil solution:aqueous solution of 4:6 and 6:4 were very similar, the hardness was significantly different. The hardness of the emulsion gel-based fat substitute with the mass fraction of oil solution of 40% was significantly greater than that of the emulsion gel-based fat substitute with the mass fraction of oil solution of 60%. This was due to the fact that the continuous phase changed, the hardness of the oleogel in the outer phase was obviously smaller than that of the hydrogel, and thus the hardness of the water-in-oil emulsion gel-based fat substitute decreased significantly when the phase change occurred.

Table 1 shows the contents of trans fatty acids and saturated fatty acids in the emulsion gel-based fat substitutes. It can be seen from table 1 that compared with commercially available margarine, the emulsion gel-based fat substitute contained no-trans fatty acids and much less saturated fatty acids than commercially available margarine (45.34%), meeting the requirement of healthy diet for consumers.

TABLE 1
Contents of trans fatty acids and saturated fatty acids in
the emulsion gel-based fat substitutes

| Oil solution: | Trans fatty | Saturated fatty |
|---|---|---|
| Aqueous solution | acids (%) | acids (%) |
| 2:8 | 0 | 3.45 |
| 4:6 | 0 | 7.30 |
| 5:5 | 0 | 8.62 |
| 6:4 | 0 | 9.15 |
| 8:2 | 0 | 13.79 |
| Commercially | 0.19 | 45.34 |
| available margarine | | |

### Comparative Example 1

The product was obtained according to the method in Example 1, except for adjusting the gelatin in step (2) of Example 1 as arabic gum.

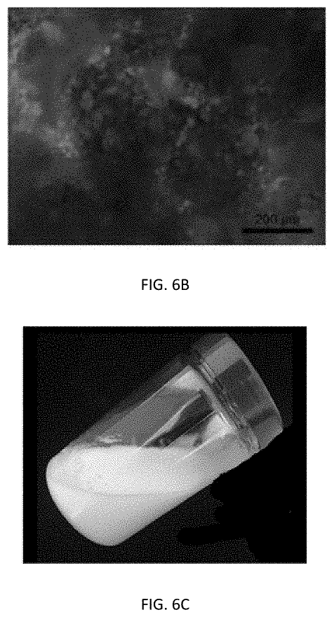

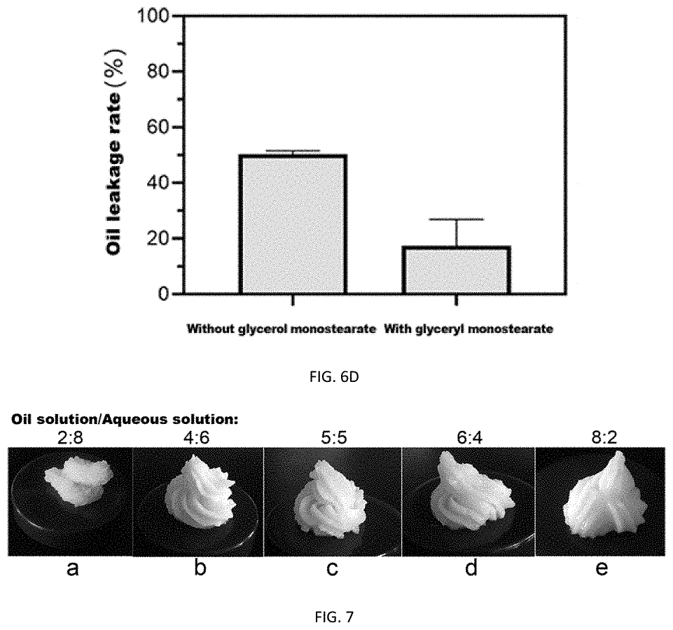

The test showed that the product did not have the structure of oil-in-water emulsion, and could not form an oil-in-water emulsion gel, as shown in FIG. 6A.

### Comparative Example 2

The product was obtained according to the method in Example 2, except for adjusting the glycerol monostearate in step (1) of Example 2 as bee wax.

The test showed that the product did not have the structure of water-in-oil emulsion, and could not form a water-in-oil emulsion gel, as shown in FIG. 6B.

### Comparative Example 3

The product was obtained according to the method in Example 1, except for adjusting the amount of gelatin in step (1) of Example 2 as 2 parts.

After the obtained product was placed at an angle in a transparent bottle, it was found that the product was extremely mobile and a gel could not be formed, as shown in FIG. 6C.

### Comparative Example 4

The product was obtained according to the method in Example 1, except for omitting the addition of glycerol monostearate in Example 1.

The obtained product was centrifuged at 10,000 rpm for 10 min for centrifugal degreasing, and the oil leakage rate of the product was higher than that of the emulsion gel prepared by adding 2 parts of glycerol monostearate in Example 1, as shown in FIG. 6D.

### Comparative Example 5

The product was obtained according to the method in Example 1, except for adjusting the ethyl cellulose in step (1) of Example 1 as polyglycerol fatty acid ester.

The products were tested for performance, and the results were as follows:

The obtained product had an elastic modulus of 85 Pa and a viscous modulus of 120 Pa, i.e., the viscous modulus was greater than the elastic modulus, had a certain fluidity, did not have semi-solid properties, and could not be used as a substitute for solid fat.

### Comparative Example 6

The product was obtained according to the method in Example 1, except for adjusting the amount of ethyl cellulose in step (1) of Example 1 as 0, 2, and 4 parts, while adding 10, 8, and 6 parts of soybean oil.

The products were tested for performance, and the results were shown in table 2 below:

TABLE 2
Test results for Comparative Example 6

| Amount of ethyl | Elastic modulus | Viscous modulus |
|---|---|---|
| cellulose | (Pa) | (Pa) |
| 0 | 12 | 23 |
| 2 parts | 127 | 163 |
| 4 parts | 1023 | 997 |

It can be seen from table 2 that the obtained products had a viscous modulus greater than or close to the elastic modulus, a small modulus and a fluidity, and did not have obvious semi-solid characteristics, and could not be used as a substitute for solid fat.

### Example 5 Use in Flower Shaped Cream

A method for preparing an emulsion gel-based fat substitute for the flower shaped cream includes the steps of:

(1) 10 parts of ethyl cellulose and 2 parts of glycerol monostearate were weighed and dissolved in 88 parts of soybean oil at 150° C., stirred for 10 min and then placed in a water bath at 70° C. to obtain an oil solution;

(2) 10 parts of gelatin were dissolved in 90 parts of hot water at 70° C. and stirred for 10 min to obtain an aqueous solution; and

(3) the oil solution of step (1) and the aqueous solution of step (2) were mixed evenly according to the mass ratio of 2:8, 4:6, 5:5, 6:4, and 8:2, and the mixed solution was emulsified for 2 min at a rate of 10,000 rpm using a high-speed homogenizer to obtain an emulsion; and then the obtained emulsion was stirred at room temperature at a low speed of 400 rpm until gelling the system to obtain an emulsion gel-based fat substitute.

The emulsion gel-based fat substitute was subjected to the flower shaped cream using metal icing head and plastic icing bag, and the obtained the flower shaped cream was shown in FIG. 7. When the ratio of oil solution to aqueous solution was 4:6, the decorating effect was most satisfactory and the flower shaped cream was uniform and stable in structure. Therefore, the oil-in-water emulsion gel-based fat substitute with the ratio of oil solution to aqueous solution of 4:6 had a better effect in food decorating application with good formability and could be used as a substitute for traditional plastic fat in food decorating.

### Example 6 Use in 3D Printing

A method for preparing an emulsion gel-based fat substitute for 3D printing includes the steps of:

(1) 10 parts of ethyl cellulose and 2 parts of glycerol monostearate were weighed and dissolved in 88 parts of soybean oil at 150° C., stirred for 10 min and then placed in a water bath at 70° C. to obtain an oil solution;

(2) 10 parts of gelatin were dissolved in 90 parts of hot water at 70° C. and stirred for 10 min to obtain an aqueous solution; and

(3) the oil solution of step (1) and the aqueous solution of step (2) were mixed evenly according to the mass ratio of 2:8, 4:6, 5:5, 6:4, and 8:2, and the mixed solution was emulsified for 2 min at a rate of 10,000 rpm using a high-speed homogenizer to obtain an emulsion; and then the obtained emulsion was stirred at room temperature at a low speed of 400 rpm until gelling the system to obtain an emulsion gel-based fat substitute.

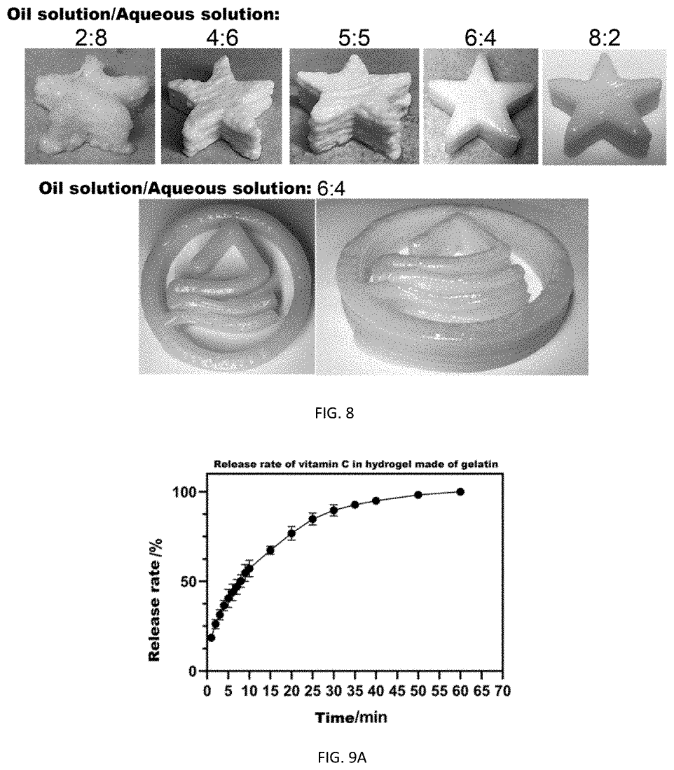

The emulsion gel-based fat substitute was printed using a 3D food printer, in which the diameter of the printing needle was 1.55 mm, and the temperature and rate of printing were 25° C. and 25 mm/s, respectively. The effect of 3D printing was shown in FIG. 8. When the ratios of the oil solution to the water solution were 6:4 and 8:2, the printing effects were more ideal, and the printed structure was delicate and smooth. Therefore, the water-in-oil emulsion gel-based fat substitutes with the ratios of the oil solution to the aqueous solution of 6:4 and 8:2 had better effects in 3D printing application, good material fluidity and outstanding plasticity, and could be used to customize various new foods in 3D food printing.

### Example 7 Use in the Controlled Release of Functional Active Components

A method for preparing an emulsion gel-based fat substitute for controlled release of functional active components includes the steps of:

(1) 10 parts of ethyl cellulose and 2 parts of glycerol monostearate were weighed and dissolved in 88 parts of soybean oil at 150° C., stirred for 10 min and then placed in a water bath at 70° C. to obtain an oil solution;

(2) 10 parts of gelatin were dissolved in 89 parts of hot water at 70° C., 1 part of vitamin C was added and stirred for 10 min to obtain an aqueous solution; and

(3) the oil solution of step (1) and the aqueous solution of step (2) were mixed evenly according to the mass ratio of 4:6, 5:5, and 6:4, and the mixed solution was emulsified for 2 min at a rate of 10,000 rpm using a high-speed homogenizer to obtain an emulsion; and then the obtained emulsion was stirred at room temperature at a low speed of 400 rpm until gelling the system to obtain a water-in-oil emulsion gel-based fat substitute.

### Comparative Example 7

10 parts of gelatin were dissolved in 89 parts of hot water at 70° C., 1 part of vitamin C was added to the aqueous solution and stirred uniformly for 10 min and then cooled to room temperature to obtain a vitamin C-embedded hydrogel.

5 g of the vitamin C-embedded emulsion gel-based fat substitute in Example 7 and 5 g of the hydrogel in Comparative Example 7 were added to 100 mL of phosphate buffer, placed in a shaker and shaken at 400 rpm. A small amount of buffer was removed every few minutes. The absorption intensity was measured at 285 nm using a UV spectrophotometer to calculate the released amount of vitamin C.

### Example 8 Use in the Controlled Release of Functional Active Components

A method for preparing an emulsion gel-based fat substitute for controlled release of functional active components includes the steps of:

(1) 10 parts of ethyl cellulose and 2 parts of glycerol monostearate were weighed and dissolved in 87 parts of soybean oil at 150° C., 1 part of vitamin E was added, stirred for 10 min and then placed in a water bath at 70° C. to obtain an oil solution;

(2) 10 parts of gelatin were dissolved in 90 parts of hot water at 70° C. and stirred for 10 min to obtain an aqueous solution; and

(3) the oil solution of step (1) and the aqueous solution of step (2) were mixed evenly according to the mass ratio of 1:9, 2:8, and 3:7, and the mixed solution was emulsified for 2 min at a rate of 10,000 rpm using a high-speed homogenizer to obtain an emulsion; and then the obtained emulsion was stirred at room temperature at a low speed of 400 rpm until gelling the system to obtain a vitamin E-embedded oil-in-water emulsion gel-based fat substitute.

### Comparative Example 8

10 parts of ethyl cellulose and 2 parts of glycerol monostearate were dissolved in 87 parts of soybean oil at 150° C., stirred for 10 min, and then placed in a water bath at 70° C. 1 part of vitamin E was added to the oil solution, stirred uniformly for 10 min, and then cooled to room temperature to obtain a vitamin E-embedded oleogel.

5 g of the vitamin E-embedded emulsion gel-based fat substitute in Example 8 and 5 g of the oleogel in Comparative Example 8 were added to 100 mL of phosphate buffer, placed in a shaker and shaken at 400 rpm. A small amount of the sample was removed every few minutes, which was added to 50 mL of is opropanol. The absorption intensity was measured at 297.9 nm using a UV spectrophotometer to calculate the released amount of vitamin E.

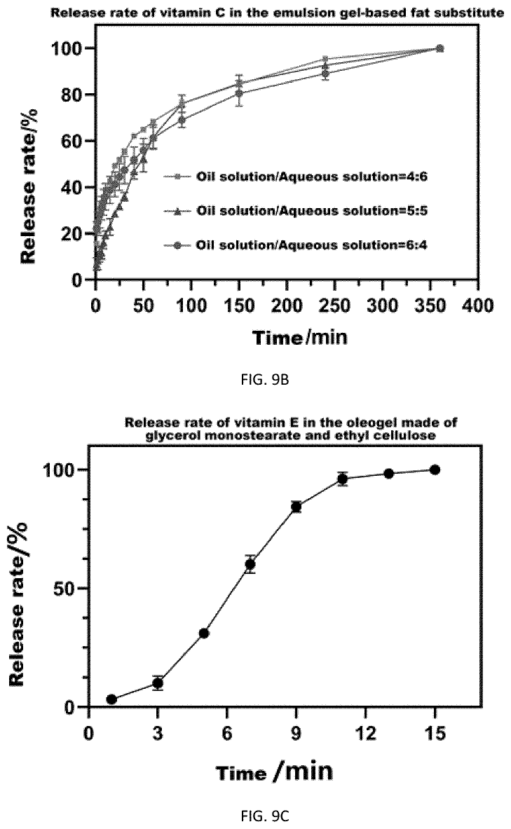

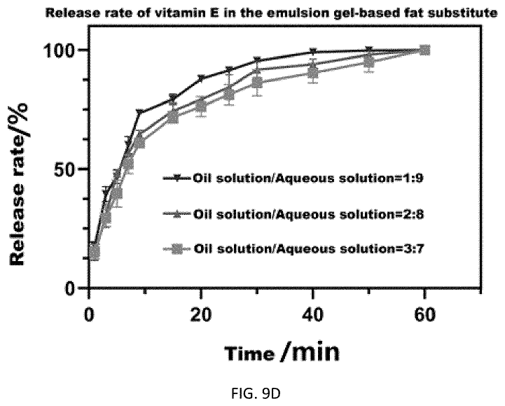

The release rates of vitamin C and vitamin E were shown in FIGS. 9A, 9B, 9C, and 9D. The emulsion gel-based fat substitute significantly affected the slow release of vitamin C, in which the time required for the complete release of vitamin C was 350 min, while the release of vitamin C was completed after 60 min when using vitamin C embedded by hydrogel made of gelatin alone. The release of vitamin E was basically completed after 15 min when using vitamin E embedded by oleogel alone, while the time required for the complete release of vitamin E in emulsion gel-based fat substitute was greater than 45 min, indicating that the controlled release effect of emulsion gel-based fat substitutes on water-soluble vitamin C and fat-soluble vitamin E was significantly better than the embedding and slow release effect of hydrogel or oleogel alone. Therefore, the embedding and slow release effect of emulsion gel-based fat substitutes facilitates better absorption of functional nutrient components in vivo or in vitro.

What is claimed is:

1. A method for preparing an emulsion gel-based fat substitute with adjustable phase change, comprising the steps of:
(1) dissolving an oil-soluble polysaccharide and an oil-soluble small molecule gelling agent in a vegetable oil to obtain an oil solution;
(2) dissolving a water-soluble large molecular gelling agent in water to obtain an aqueous solution; and
(3) evenly mixing the oil solution in step (1) and the aqueous solution in step (2) for emulsification to obtain an emulsion; and then gelling to obtain an emulsion gel-based fat substitute,
wherein when the mass percentage of the oil solution in the emulsion in step (3) is less than 44% and more than 0, an oil-in-water emulsion gel-based fat substitute is obtained; when the mass percentage of the oil solution in the emulsion in step (3) is more than 44% and less than 100%, a water-in-oil emulsion gel-based fat substitute is obtained; and when the mass percentage of the oil solution in the emulsion in step (3) is equal to 44%, a semi-bicontinuous emulsion gel-based fat substitute is obtained.

2. The method according to claim 1, where in the water-soluble large molecular gelling agent in step (2) is one or more of gelatin, hydroxypropyl methylcellulose, methylcellulose, and konjac gum.

3. The method according to claim 1, where in the mass concentration of the water-soluble large molecular gelling agent in the water in step (2) is 4% to 10%.

4. The method according to claim 1, wherein the oil-soluble polysaccharide in step (1) is one or both of ethyl cellulose and chitin.

5. The method according to claim 1, wherein the mass concentration of the oil-soluble polysaccharide in the vegetable oil in step (1) is 5% to 10%.

6. The method according to claim 1, wherein the oil-soluble small molecule gelling agent in step (1) is one or more of mono- and diglycerides fatty acid, polyglycerol fatty acid ester, sodium stearoyl lactate, and sucrose fatty acid esters.

7. The method according to claim 1, wherein the mass concentration of the oil-soluble small molecule gelling agent in the vegetable oil in step (1) is 1% to 5%.

8. The method according to claim 1, wherein the emulsification in step (3) is a high-speed shearing at 65-80° C. and 5000-15000 rpm for 1-5 minutes.

9. The method according to claim 1, wherein the gel in step (3) is obtained by stirring the obtained emulsion at 15-30° C. and 100-1000 rpm.

10. The method according to claim 1, wherein the method for preparing an emulsion gel-based fat substitute with adjustable phase change comprises the steps of:
(1) dissolving the oil-soluble polysaccharide and the oil-soluble small molecule gelling agent in the vegetable oil at 130-180° C. to obtain the oil solution;
(2) dissolving the water-soluble large molecular gelling agent in the water at 65-80° C. to obtain the aqueous solution; and
(3) evenly mixing the oil solution in step (1) and the aqueous solution in step (2), and emulsifying via a high-speed shearing at 65-80° C. and 5000-15000 rpm for 1-5 minutes to obtain the emulsion; and then stirring and gelling at 15-30° C. and 100-1000 rpm to obtain the emulsion gel-based fat substitute.

11. The method according to claim 1, where in the vegetable oil in step (1) is one or more of peanut oil, soybean oil, sunflower oil, rapeseed oil, corn oil, tea seed oil, sesame oil, olive oil, wheat germ oil, palm oil, hemp seed oil, coconut oil, palm kernel oil, and coconut kernel oil.

12. The method according to claim 1, wherein when the mass ratio of the oil solution to the aqueous solution in step (3) is 2:8 to 4:6, the oil-in-water emulsion gel-based fat substitute is obtained; and when the mass ratio of the oil solution to the aqueous solution in step (3) is 5:5 to 8:2, the water-in-oil emulsion gel-based fat substitute is obtained.

13. The oil-in-water emulsion gel-based fat substitute and the water-in-oil emulsion gel-based fat substitute prepared by the method according to claim 1.

14. A 3D printing method, which uses water in oil lotion gel fat substitute as an edible solid material for 3D printing.

15. The method according to claim 14, wherein in the method of preparing water in oil lotion gel based fat substitute, the mass ratio of the oil solution to the aqueous solution is 6:4 to 8:2.

16. A method for embedding controlled release water-soluble active substances and fat soluble active substances, which uses oil-in-water gel-based fat substitute or the water-in-oil emulsion gel-based fat substitute as a carrier in the embedding and controlled-release of a water-soluble active substance and a fat-soluble active substance.
